(19) United States
(12) Patent Application Publication
(10) Pub. No.: US 2012/0165609 A1
(43) Pub. Date: Jun. 28, 2012

(54) BENDING DEVICE

(76) Inventors: Li Sheng LIU, Shenzhen (CN); Yong Qing Qi, Shenzhen (CN)

(21) Appl. No.: 13/336,531

(22) Filed: Dec. 23, 2011

(30) Foreign Application Priority Data
Dec. 23, 2010 (CN) 201010608019.6

(51) Int. Cl. A61B 1/00

(52) U.S. Cl. 600/146

(57) ABSTRACT

A bending device includes a bendable unit which has a bendable body extending in a direction and a soft pipe extending in the same direction and fixed to the bendable body. The soft pipe is stretchable in said direction relative to the bendable body in response to changes of pressure inside the soft pipe, which results in the bendable body and the soft pipe bending.

### CROSS REFERENCE TO RELATED APPLICATIONS

This non-provisional patent application claims priority under 35 U.S.C. §119(a) from Patent Application No. 201010608019.6 filed in The People's Republic of China on Dec. 23, 2010.

### FIELD OF THE INVENTION

This invention relates to a bending device, and in particular to a bending device which can be adapted for use in medical instruments and endoscopes, although other applications are envisaged.

### BACKGROUND OF THE INVENTION

Bending devices are widely used in many fields, such as robotic arms, endoscopes, inspection devices and medical instruments such as gastric feeding devices, etc.

A typical bending device comprises a bendable rod, a plurality of spaced laminations fixed on the bendable rod, and wires extending through the laminations. Each lamination defines a central hole in the center thereof and openings at the periphery thereof. The bendable rod extends through the central holes of the laminations. The wires each extend through a corresponding opening of the laminations with one end thereof fixed to the outer most one of the laminations. When one wire is drawn the bending device is bent accordingly.

However, the above bending device has a complicated structure, big size and high cost. Hence there is a desire for an improved bending device.

### SUMMARY OF THE INVENTION

Accordingly, in one aspect thereof, the present invention provides a bending device comprising a bendable unit, wherein the bendable unit comprises a bendable body extending in a direction and a soft pipe extending in said direction and fixed to the bendable body, the soft pipe being stretchable in said direction relative to the bendable body, whereby the bendable body and the soft pipe bend together in response to a change of pressure inside the soft pipe.

Preferably, a control unit communicates with the soft pipe via a conduit in order to control the change of pressure inside the soft pipe.

Preferably, the control unit comprises pumps or capsules containing fluid therein, the pumps or capsules being in communication with the soft pipe via the conduit.

Preferably, the bendable body is made of non-stretchable material such that the bendable body is easy to bend but difficult to stretch in said extending direction thereof, and the soft pipe is made of gas-impermeable material.

Preferably, the bendable body defines a void in said extending direction thereof.

Preferably, at least two soft pipes are fixed to the bendable body and arranged in a circumferential direction of the bendable body.

Preferably, at least three soft pipes are fixed to the bendable body and arranged in a circumferential direction of the bendable body, the bendable body comprising a central portion and a plurality of claws extending outwardly from the central portion, each soft pipe being fixed between two adjacent claws and adjacent soft pipes being separated by a corresponding claw.

Preferably, each claw defines a plurality of spaced notches/slits/slots in an outer surface thereof, the notches/slits/slots being separately arranged along said extending direction of the bendable body.

Preferably, an optical fiber passes through the inside of the bendable body or the soft pipe.

Preferably, a constraining layer is provided on an outer surface of the soft pipe to constrain radial expansion of the soft pipe.

Preferably, the constraining layer is made of non-stretchable band/strip/wire wound helically around the soft pipe.

According to a second aspect, the present invention provides a medical apparatus comprising an inserting unit and a bending device as defined above, wherein the bending device is disposed at a distal end of the inserting unit.

Preferably, the medical apparatus is a gastric feeding device and further comprises at least one nutrient feeding conduit and/or medicine feeding conduit passing through the inside of the bendable body or the soft pipe.

According to a further aspect, the present invention also provides a mechanical joint incorporating a bending device as defined above.

### BRIEF DESCRIPTION OF THE DRAWINGS

Preferred embodiments of the invention will now be described, by way of example only, with reference to figures of the accompanying drawings. In the figures, identical structures, elements or parts that appear in more than one figure are generally labeled with a same reference numeral in all the figures in which they appear.

Dimensions of components and features shown in the figures are generally chosen for convenience and clarity of presentation and are not necessarily shown to scale. The figures are listed below.

FIG. 1 shows a bendable unit of a bending device in accordance with a first embodiment of the present invention;

FIG. 2 shows the bendable unit of FIG. 1 in a bent position;

FIG. 3 shows a bendable unit of a bending device in accordance with a second embodiment of the present invention;

FIG. 4 is an axial sectional view of the bendable unit of FIG. 3 in a bent position;

FIG. 5 shows a bendable unit of a bending device in accordance with a third embodiment of the present invention;

FIG. 6 shows an enlarged portion of the bendable unit of FIG. 5;

FIG. 7 shows an enlarged portion of a soft pipe of the bendable unit of FIG. 6;

FIG. 8 shows a control unit of the bending device in accordance with the third embodiment of the present invention;

FIG. 9 shows a bendable unit of a bending device in accordance with a fourth embodiment of the present invention;

FIG. 10 shows a bendable unit of a bending device in accordance with a fifth embodiment of the present invention;

FIG. 11 shows a gastric feeding device in accordance with a sixth embodiment of the present invention;

FIG. 12 is an enlarged view of the bendable unit which is enclosed by a flexible casing;

FIG. 13 shows the bendable unit of FIG. 12 with the flexible casing removed;

FIG. 14 is a radial cross section of the bendable unit of FIG. 13 along line A-A;

FIG. 15 shows an enlarged bendable body of the bendable unit of FIG. 13;

FIG. 16 is a cross section view of FIG. 15 viewed along line B-B;

FIG. 17 is a cross section view of FIG. 15 viewed along line C-C;

FIG. 18 shows a bendable unit of an endoscope in accordance with a seventh embodiment of the present invention; and

FIG. 19 show a soft pipe with a protective layer provided at the outer surface thereof.

### DETAILED DESCRIPTION OF THE PREFERRED EMBODIMENTS

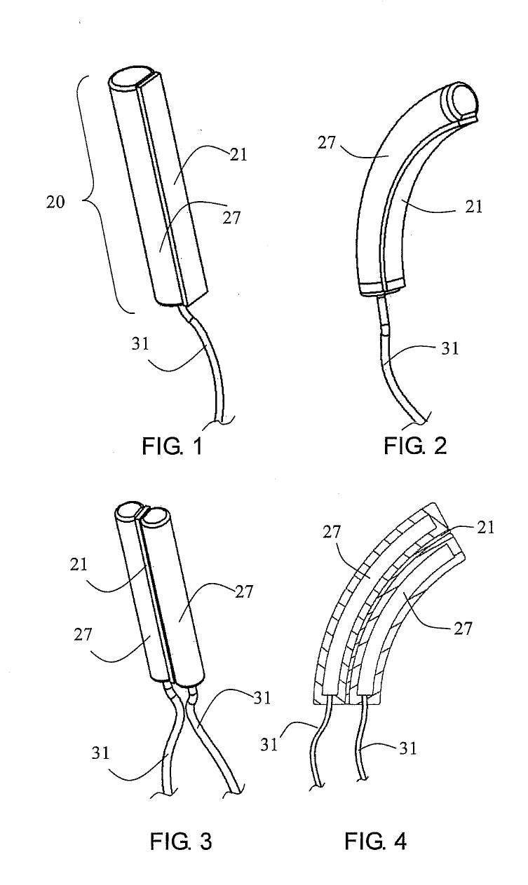

FIGS. 1 and 2 show a bending device according to a first embodiment of the present invention. The bending device comprises a bendable unit 20 which comprises a bendable body 21 and a soft pipe 27 fixed to the bendable body 21.

The bendable body 21 may be an elongated strip/plate/rod made of any suitable material such that the bendable body 21 is easy to bend but difficult to stretch in the extending direction thereof. In this embodiment, the bendable body 21 is made of rubber.

The soft pipe 27 has a hollow cylindrical configuration with sealed ends. Preferably, the soft pipe 27 is fixed to the bendable body 21 along the whole longitudinal direction thereof. The axial direction of the soft pipe 27 is substantially parallel to the longitudinal direction of the bendable body 21. The soft pipe 27 and the bendable body 21 have substantially the same length. The soft pipe 27 may be made of any suitable gas-impermeable material such that the soft pipe 27 has good stretch ability and bend ability. In this embodiment, the soft pipe 27 is made of silicone.

The soft pipe 27 may be fixed to the bendable body 21 by adhesive or integrally formed with the outer surface of the bendable body 21.

As shown in FIG. 2, the bending device further comprises a conduit 31 which communicates with the soft pipe 27 in order to supply/withdraw fluid to/from the soft pipe 27. The fluid may be liquid or gas. When the conduit 31 feeds fluid to the soft pipe 27 to increase the pressure inside of the soft pipe 27, the soft pipe 27 extends relative to the bendable body 21 which results in the soft pipe 27 bending towards the bendable body 21. Consequently, the bendable body 21 is bent together with the soft pipe 27. The bending device of this embodiment is suitable for apparatus which needs bending movement in one direction, such as robot arms/hands.

FIGS. 3 and 4 show a bi-directional bending device according to a second embodiment of the present invention. The bending device comprises a pair of soft pipes 27 fixed to opposite sides of a bendable body 21. Each soft pipe 27 communicates with a conduit 31. When one of the soft pipes 27 is fed with fluid to increase the pressure inside thereof, the soft pipe 27 bends toward the other soft pipe 27. Thus, the bending device of the second embodiment is capable of bending alternatively in one of two opposite directions.

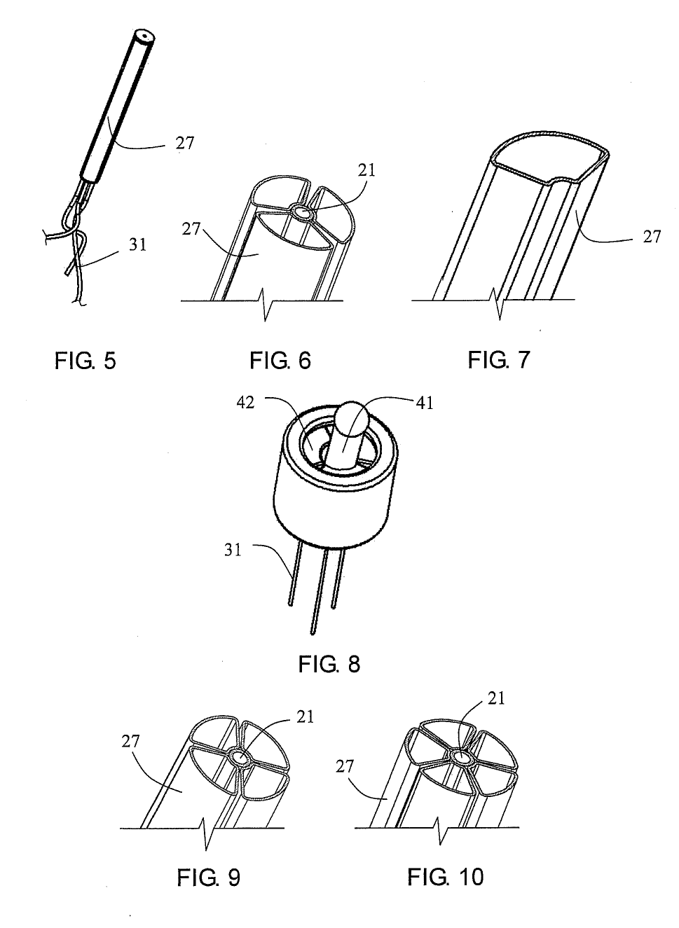

FIGS. 5 to 7 show a multi-directional bending device according to a third embodiment of the present invention. The bending device comprises a round bendable body 21 and three soft pipes 27 fixed to the outer surface of the bendable body 21. The soft pipes 27 are disposed equally spaced about the circumference of the bendable body 21. The bending device of the third embodiment is capable of bending in multi directions.

Preferably, the soft pipe 27 has a sector-shaped cross section.

Preferably, the bendable body 21 is a hollow pipe having a void therein.

The multi-directional bending device further comprises a control unit as shown in FIG. 8, which comprises a handle 41 and three soft capsules 42 arranged laterally of the handle 41. Each soft capsule 42 communicates with a corresponding soft pipe 27 via a respective conduit 31. The soft capsule 42 functions as a pump to supply/withdraw fluid to/from the corresponding soft pipe 27 via the corresponding conduit 31. The handle 41 may be pressed against a soft capsule 42 which results in the corresponding soft pipe 27 which communicates with the pressed soft capsule 42 to extend relative to others to thereby cause the bendable unit to bend. Understandably, when the soft capsule 42 is released the corresponding soft pipe 27 is capable of returning to its original state. Optionally, one or two soft capsules may be pressed to a varying degree simultaneously to effect bending of the bending unit in a desired direction.

FIG. 9 shows a multi-directional bending device with four soft pipes 27. FIG. 10 shows a multi-directional bending device with five soft pipes 27. The more soft pipes 27 a bending device uses, the easier it is to control the bending direction of the bending device.

It should be noted that the number of the soft pipes 27 may be chosen according to design requirements.

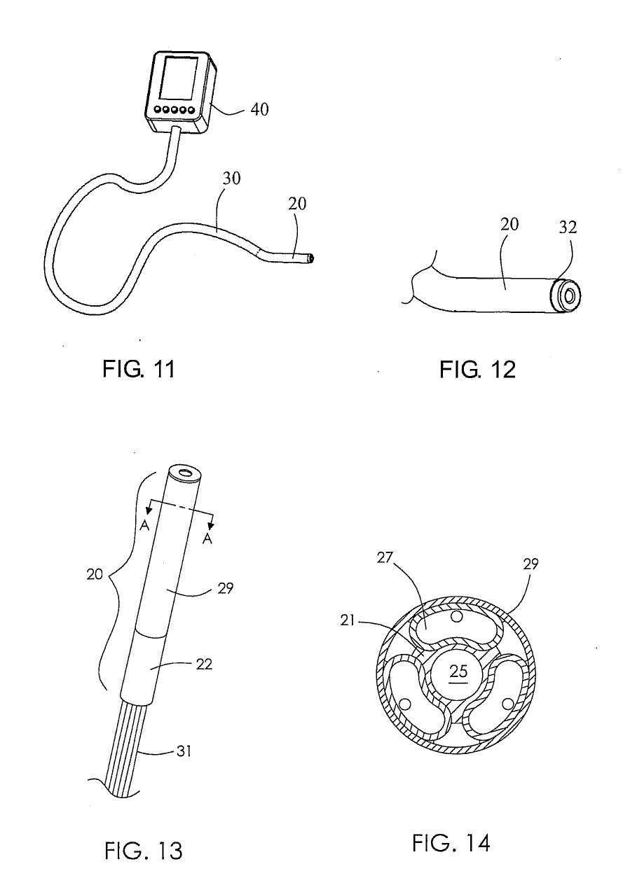

FIGS. 11 to 14 show a medical device in the form of a gastric feeding device in accordance with a sixth embodiment of the present invention. FIG. 12 is an enlarged view of the bendable unit 20 which is enclosed by a sealing cover 32. The gastric feeding device comprises a control unit 40, an inserting unit 30 and a bendable unit 20 arranged at one end of the inserting unit 30. The other end of the inserting unit 30 is connected to the control unit 40. A sealing cover 32 may be provided to enclose the inserting unit 30 and the bendable unit 20 to thereby seal the inserting unit 30 and the bendable unit 20. Preferably, the sealing cover 32 is made of flexible material with good sealing characteristics. In use, the bendable unit 20 and part of the inserting unit 30 may be inserted into an organ, such as a stomach or a duodenum of a patient, via the esophagus, for feeding or medicating the patient.

FIG. 13 shows the bendable unit 20 with the sealing cover 32 removed. FIG. 14 is a cross sectional view of the bendable unit of FIG. 13 taken along line A-A. The bendable unit 20 comprises a bendable body 21, three soft pipes 27 and a protective casing 29 enclosing the bendable body 21 and soft pipes 27. The soft pipes 27 are arranged in the circumferential direction of the bendable body 21 and fixed to the bendable body 21. The protective casing 29 is bendable and stretchable but not easy to expand in the radial direction. Thus, the protective casing 29 is capable of protecting the soft pipes 27 and preventing the soft pipes 27 from expanding in radial directions. Each soft pipe 27 is connected to a corresponding conduit 31 which may be received in the protective casing 29 and connected to the control unit 40. Other conduits 31 such as nutrient feeding conduits, medicine feeding conduits and/or optical fibers may also be enclosed in the protective casing 29.

The control unit 40 comprises a display and a plurality of buttons. The control unit functions to control the bending direction and the degree of bending of the bendable unit. The control unit may comprise pumps/capsules communicating with a container containing fluid therein and valves connected between the pumps and soft pipes 27 of the bendable unit 20.

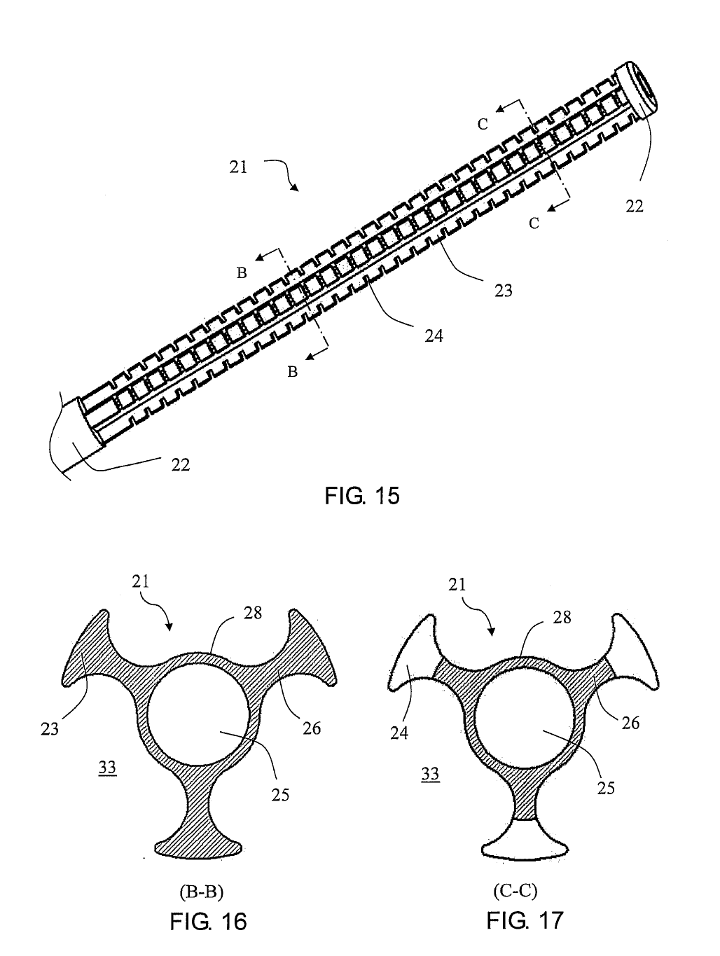

FIG. 15 shows a bendable body 21 on an enlarged scale. FIG. 16 is a cross sectional view of FIG. 15 taken along line B-B and FIG. 17 is a cross-sectional view of FIG. 15 taken along line C-C.

Referring to FIGS. 15-17, the bendable body 21 has a thin elongated configuration. Opposite ends of the bendable body 21 each have a radial size larger than that of other portions of the bendable body such that the opposite ends function as blocks 22 for locating soft pipes 27 there between. Preferably, the bendable body 21 is hollow, having a void 25 extending along the axis thereof. The void 25 serves to increase the flexibility of the bendable body 21. Understandably, nutrient feeding conduits, medicine feeding conduits and optical fibers may be received in the void 25 of the body 21. The body 21 comprises a central portion 28 and a plurality of claws 23 extending radially and outwardly from the central portion. Each claw 23 has a neck 26 connected to the central portion 28. The void 25 is defined in the central portion 28. A receiving space 33 is formed between adjacent claws 23 and the central portion 28. Each soft pipe 27 is fixed in a corresponding space 33 in close contact with the central portion 28 and claws 23 to prevent radial deformation when the soft pipe 27 is supplied with fluid. As shown in FIGS. 15 and 17, each claw 23 has a plurality of notches/slits/slots 24 in the outer surface thereof. The notches/slits/slots 24 are separately disposed along the extending direction of the body 21. The notches/slits/slots 24 improve bendability of the bendable body 21.

Preferably, the depth of the notches/slits 24 in the radial direction of the body 21 is less than that of the claws 23.

Alternatively, the depth of the notches/slits/slots 24 in the radial direction of the body 21 may be equal to or slightly larger than that of the claws 23.

In the embodiment of the present invention, the bendable unit 20 of the gastric feeding device has good bendability. Therefore, the inserting unit 30 is capable of easily maneuvering around corners or through small voids in the bodies of patients.

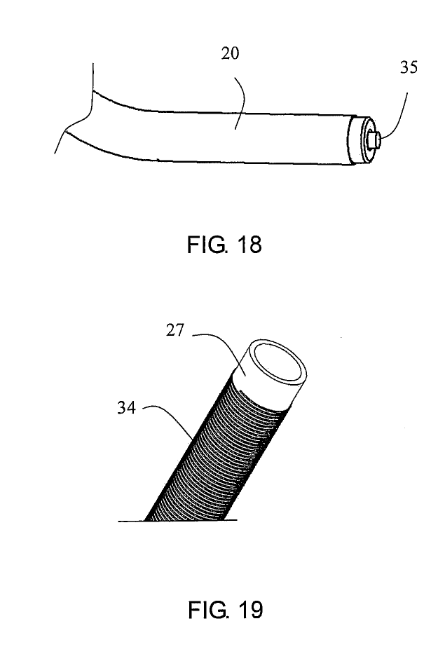

FIG. 18 shows a bendable unit of an inspection device such as an endoscope instrument in accordance with a seventh embodiment of the present invention. As shown in FIG. 11, the endoscope instrument comprises a control unit 40, an inserting unit 30 and a bendable unit 20. As shown in FIG. 18, the endoscope further comprises at least one optical fiber or bundle of optical fibers 35 received in the void 25 of the bendable unit 20 for transmitting light and/or images. Illuminating apparatus and image forming apparatus connecting with the fibers may be installed at the distal end of the bendable unit 20 for illuminating and forming images of organs to be inspected.

FIG. 19 shows a constraining layer 34 provided on the outer surface of the soft pipe 27 to prevent the soft pipe 27 from expanding radially. Preferably, the constraining layer 34 is formed by a cotton wire wound helically around the soft pipe 27. When the pressure inside the soft pipe 27 increases, the soft pipe 27 extends in the axial direction thereof. The constraining layer 34 formed by the cotton wire extends along with the soft pipe 27 and thus decreases in diameter and limits radial expansion of the soft pipe 27.

Alternatively, the constraining layer 34 may be formed by a helical spring.

It should be noted that the bendable unit 20 of the present invention can be used in other fields such as mechanical joints, for example robot arms/hands for industry, as well as for medical instruments and endoscope inspection instruments.

In the description and claims of the present application, each of the verbs “comprise”, “include”, “contain” and “have”, and variations thereof, are used in an inclusive sense, to specify the presence of the stated item but not to exclude the presence of additional items.

Although the invention is described with reference to one or more preferred embodiments, it should be appreciated by those skilled in the art that various modifications are possible. Therefore, the scope of the invention is to be determined by reference to the claims that follow.

1. A bending device comprising a bendable unit,
wherein the bendable unit comprises a bendable body extending in a direction and a soft pipe extending in said direction and fixed to the bendable body, the soft pipe being stretchable in said direction relative to the bendable body, whereby the bendable body and the soft pipe bend together in response to a change of pressure inside the soft pipe.

2. The bending device of claim 1, further comprising a control unit which communicates with the soft pipe via a conduit in order to control the change of the pressure inside of the soft pipe.

3. The bending device of claim 2, wherein the control unit comprises pumps or capsules containing fluid therein, the pumps or capsules being in communication with the soft pipe via the conduit.

4. The bending device of claim 2, wherein the bendable body is made of non-stretchable material such that the bendable body is easy to bend but difficult to stretch in said extending direction thereof, and the soft pipe is made of gas-impermeable material.

5. The bending device of claim 1, wherein the bendable unit comprises at least two soft pipes fixed to the bendable body and arranged in a circumferential direction of the bendable body.

6. The bending device of claim 1, wherein the bendable body defines a void in said extending direction thereof.

7. The bending device of claim 1, wherein the bendable unit comprises at least three soft pipes fixed to the bendable body and arranged in a circumferential direction of the bendable body, the bendable body comprising a central portion and a plurality of claws extending outwardly from the central portion, each soft pipe being fixed between two adjacent claws and adjacent soft pipes being separated by a corresponding claw.

8. The bending device of claim 7, wherein each claw defines a plurality of spaced notches/slits/slots in an outer surface thereof, the notches/slits/slots being separately arranged along said extending direction of the bendable body.

9. The bending device of claim 7, further comprising a protective casing enclosing the bendable body and soft pipes in order to limit radial expansion of the soft pipes.

10. The bending device of claim 1, wherein a constraining layer is provided on an outer surface of the soft pipe to constrain radial expansion of the soft pipe.

11. The bending device of claim 10, wherein the constraining layer is made of non-stretchable band/strip/wire wound helically around the soft pipe.

12. A medical apparatus comprising an inserting unit and the bending device of claim 1, wherein the bending device is disposed at the distal end of the inserting unit.

13. The medical apparatus of claim 12, further comprising a control unit, the control unit comprising a pump/capsule communicated with the soft pipe of the bendable unit via a conduit which passes through the inserting unit.

14. The medical apparatus of claim 13, further comprising an optical fiber passing inside of the bendable body or the soft pipe.

15. The medical apparatus of claim 13, wherein the medical apparatus is a gastric feeding device which further comprises at least one nutrient feeding conduit and/or medicine feeding conduit passing through the inside of the bendable body or the soft pipe.

16. A mechanical joint incorporating the bending device of claim 1.
